(19) United States
(12) Patent Application Publication
(10) Pub. No.: US 2023/0256420 A1
(43) Pub. Date: Aug. 17, 2023

(54) SHAPED CATALYST BODY FOR THE PRODUCTION OF ETHYLENE OXIDE

(71) Applicant: BASF SE, Ludwigshafen am Rhein (DE)

(72) Inventors: Sung Yeun CHOI, Ludwigshafen am Rhein (DE); Andrey KARPOV, Ludwigshafen am Rhein (DE); Christian WALSDORFF, Ludwigshafen am Rhein (DE); Patrick HUBACH, Ludwigshafen am Rhein (DE)

(21) Appl. No.: 18/011,542

(22) PCT Filed: Jun. 24, 2021
(86) PCT No.: PCT/EP2021/067395

§ 371 Date: Dec. 20, 2022

(30) Foreign Application Priority Data
Jun. 26, 2020 (EP) 20182577.5

(51) Int. Cl. B01J 23/68; B01J 35/10; B01J 21/04; B01J 6/00; C07C 29/50

(52) U.S. Cl. CPC B01J 23/688; B01J 35/1014; B01J 23/68; B01J 21/04; B01J 35/1009; B01J 6/001; C07C 29/50

(57) ABSTRACT

A shaped catalyst body for producing ethylene oxide by gas-phase oxidation of ethylene, having a BET surface area in the range of 2 to 20 m2/g and comprising silver and a rhenium promotor deposited on a porous alpha-alumina catalyst support, characterized in that the support has a calcination history of at least 1460° C. The catalyst support has a high surface area and little ethylene oxide isomerization and/or decomposition activity. The invention further relates to a porous alpha-alumina catalyst support having a BET surface area of 1.7 to 10 m2/g, the porous alpha-alumina catalyst support being obtainable by a) preparing a precursor material comprising a transition alumina and/or an alumina hydrate; b) forming the precursor material into shaped bodies; and c) calcining the shaped bodies at a temperature of 1460° C. to 1700° C. to obtain the porous alpha-alumina support. The invention also relates to a process for producing ethylene oxide by gas-phase oxidation of ethylene, comprising reacting ethylene and oxygen in the presence of a shaped catalyst body as described above.

The present invention relates to a shaped catalyst body for producing ethylene oxide by gas-phase oxidation of ethylene, a porous alpha-alumina catalyst support, and a process for producing ethylene oxide by gas-phase oxidation of ethylene.

Ethylene oxide is produced in large volumes and is primarily used as an intermediate in the production of several industrial chemicals. In the industrial oxidation of ethylene to ethylene oxide, heterogeneous catalysts comprising silver deposited on a porous support are typically used. To carry out the heterogeneously catalyzed gas-phase oxidation, a mixture of an oxygen-comprising gas, such as air or pure oxygen, and ethylene is generally passed through a plurality of tubes which are arranged in a reactor in which a packing of shaped catalyst bodies is present. Highly selective ethylene oxide catalysts typically comprise porous alpha-alumina supports impregnated with silver and a rhenium promoter.

The production of ethylene oxide by the vapor-phase catalytic oxidation of ethylene with molecular oxygen in the presence of silver catalyst is, however, susceptible to simultaneously entailing side reactions, which are represented by a so-called complete combustion. Another reaction affecting the yield of ethylene oxide is the isomerization of ethylene oxide to acetaldehyde. It is also envisaged that the ethylene oxide formed by isomerization of ethylene oxide may be further oxidized to yield carbon dioxide and water.

The extent to which these entailing side reactions occur depends on the quality of the catalyst in use. The catalyst quality, in turn, depends on the dispersion of the active silver metal, the presence of promotors, if any, and the inherent propensity of the support to isomerize or decompose ethylene oxide. This explains why the improvement of catalyst supports has constituted a major research task.

It is desirable that the silver be relatively uniformly dispersed on the interior and exterior surfaces of the support. More specifically, highly dispersed fine silver particles allow for attaining improved activity and selectivity and for maintaining high activity and selectivity levels over time. High surface area of the support allows for high dispersion of silver. However, by simply increasing the surface area of the support, the intended effect cannot automatically be obtained, because there is an influence of side-reactions that may occur on the surface of the support.

Alumina (Al₂O₃) is ubiquitous in supports and/or catalysts for many heterogeneous catalytic processes. It is well known that alumina has a number of crystalline phases such as alpha-alumina (often denoted as α-alumina or α-Al₂O₃), gamma-alumina (often denoted as γ-alumina or γ-Al₂O₃) as well as a number of alumina polymorphs. Alpha-alumina is the most stable at high temperatures, but has the lowest surface area.

Typically, α-alumina based EO catalyst supports are made by incorporating α-alumina crystals, made by calcination of alumina hydrate precursors, into an extrudate mixture with binders (SiO₂, magnesium silicate, calcium silicate, ZrO₂, TiO₂, boehmite (AlOOH), boric acid, etc.). The binders are used as a bonding material between the α-alumina crystals to enhance the mechanical strength of the aggregates and to reduce the sintering time needed to achieve such strength. However, binders may introduce undesired active sites in the α-alumina supports, for example acidic sites. Such acidic sites are believed to accelerate the undesired conversion of ethylene oxide in the gas-phase oxidation of ethylene, for example to acetaldehyde. Thus, it would be ideal to prepare supports without binders which introduce elements different than aluminum, oxygen and hydrogen. The extrudates are calcined at high temperatures to enhance the mechanical properties, control porosity, pore size, and pore size distribution.

Gamma-alumina has a very high surface area. This is generally believed to be because the alumina molecules are in a crystalline structure that is not very densely packed. Gamma-alumina constitutes a part of the series known as activated aluminas or transition aluminas, so-called because it is one of a series of aluminas that can undergo transition to different polymorphs. When gamma-alumina is heated to high temperatures, e.g., during calcination, the structure of the atoms collapses to a certain extent such that the surface area decreases. The most dense crystalline form of alumina is alpha-alumina. Nonetheless, alpha-alumina supports obtained from heat treatment of transition aluminas may exhibit a favorably large BET surface area. Transition aluminas comprise a large number of surface hydroxy groups and/or Lewis acid sites. Most of the surface acidity is eliminated from the α-alumina crystal surfaces upon conversion to α-alumina and final calcination of the extrudates.

There remains a significant need to enhance the performance of a supported catalyst by optimizing the alumina-based support structure. The support structure should have a combination of high surface area with little ethylene oxide isomerization and/or decomposition activity.

WO 2012/143557 A1 describes porous alpha-alumina supports obtained by calcining at temperatures between 1440 and 1480° C. and having BET surfaces in the range of 0.6 to 1 m²/g.

WO 2012/143559 A1 describes porous alpha-alumina supports obtained by calcining at temperatures between 1440 and 1520° C. and having BET surfaces in the range of 0.6 to 0.8 m²/g.

WO 03/072244 A1 describes porous alpha-alumina supports obtained by calcining at a temperature of 1425° C. and having BET surfaces in the range of 0.7 to 2.5 m²/g.

US 2012/0301666 A1 describes porous alpha-alumina supports obtained by calcining at temperatures between 1600 and 1750° C.

U.S. Pat. No. 5,384,302 A describes porous alpha-alumina supports obtained by calcining at a temperature of 1482° C. and having BET surfaces in the range of 1.2 to 1.5 m²/g.

It has now been found that using transition aluminas and/or hydrated aluminas as starting materials for the production of alpha-alumina catalyst supports allows for a high calcination temperature of the supports while simultaneously allowing for a high BET surface area of the supports.

The invention relates to a shaped catalyst body for producing ethylene oxide by gas-phase oxidation of ethylene, having a BET surface area of 2 to 10 m²/g and comprising silver and a rhenium promotor deposited on a porous alpha-alumina catalyst support, characterized in that the support has a calcination history of at least 1460° C., preferably 1460 to 1700° C.

According to the invention, the support has a calcination history of at least 1460° C. The term “calcination history” is understood to mean that an alpha-alumina precursor material or the alpha-alumina has been subjected to heat treatment at a temperature of at least 1460° C. This includes, e.g., situations where a precursor material extrudate has been subjected to a heat treatment in this temperature range, as well as situations where a support obtained at lower calcination temperatures has been subjected to a subsequent heat treatment in this temperature range. Preferably, the extrudate or support has a calcination history of being exposed to a heat treatment in this temperature range for at least 30 min, in particular at least 240 min.

The support preferably has a calcination history of being exposed to a temperature of 1460 to 1700° C., more preferably 1475 to 1600° C., most preferably 1490 to 1550° C.

The impact of the calcination history can be observed by subjecting alpha-aluminas with different calcination history to an ethylene oxide decomposition test. Such an ethylene oxide decomposition test comprises placing a defined amount of catalyst support bodies, optionally crushed to a specific size, in a reactor and passing a gas mixture comprising ethylene oxide and oxygen over the catalyst support at a given temperature, e.g., 150 to 300° C., and a given inlet pressure, e.g. at an inlet pressure of 1 to 25 bars, for several hours. The amounts of ethylene oxide are determined and compared at the reactor inlet and reactor outlet, and the degree of ethylene oxide decomposition is calculated therefrom.

The shaped catalyst body of the invention has a BET surface area of 2 to 20 m²/g. The BET method is a standard, well-known method and widely used method in surface science for the measurements of surface areas of solids by physical adsorption of gas molecules. The BET surface is determined according to DIN ISO 9277 herein, unless stated otherwise. The terms “BET surface area” and “surface area” are used equivalently herein, unless noted otherwise.

The shaped catalyst body preferably has a BET surface in the range of 2 to 15 m²/g, more preferably 2 to 10 m²/g, most preferably 2 to 3 m²/g.

As shown in the working examples herein, porous alpha-alumina catalyst supports calcined at high temperatures and having a high BET surface area allow for shaped catalyst bodies exhibiting favorably high selectivities and activities.

The porous alpha-alumina catalyst support is obtainable by
i) preparing a precursor material comprising a transition alumina and/or an alumina hydrate;
ii) forming the precursor material into shaped bodies; and
iii) calcining the shaped bodies at a temperature of at least 1460° C., preferably 1460 to 1700° C., to obtain the porous alpha-alumina support.

The invention also relates to a porous alpha-alumina catalyst support obtainable by this method. In the following, all embodiments relate to the porous alpha-alumina catalyst support itself as well as to the porous alpha-alumina catalyst support comprised in the shaped catalyst body of the invention.

The term “transition alumina” is understood to mean an alumina comprising a metastable alumina phase, such as a gamma-, delta-, eta-, theta-, kappa- or chi-alumina phase. Preferably, the transition alumina comprises at least 80 wt.-%, preferably at least 90 wt.-%, most preferably at least 95 wt.-%, such as 95 to 100 wt.-%, of a phase selected from gamma-alumina, delta-alumina and/or theta-alumina, based on the total weight of the transition alumina, in particular a phase selected from gamma-alumina and/or delta-alumina.

The transition alumina is typically in the form of a powder. Transition aluminas are commercially available and may be obtained via thermal dehydration of hydrated aluminum compounds, in particular hydrated aluminum hydroxides and hydrated aluminum oxy-hydroxides. Suitable hydrated aluminum compounds include naturally occurring and synthetic compounds, such as aluminum trihydroxides like gibbsite, bayerite and nordstrandite, or aluminum oxy-monohydroxides like boehmite, pseudoboehmite and diaspore.

By progressively dehydrating hydrated aluminum compounds, lattice rearrangements are affected. For example, boehmite can be converted to gamma-alumina at about 450° C., gamma-alumina can be converted to delta-alumina at about 750° C., and delta-alumina can be converted to theta-alumina at about 1000° C. When heating at above 1000° C., transition aluminas are converted to alpha-alumina.

It is believed that the morphological properties of the resulting transition aluminas are primarily dependent on the morphological properties of the hydrated aluminum compounds from which they are derived. Accordingly, Busca, “The Surface of Transitional Aluminas: A Critical Review”, Catalysis Today, 226 (2014), 2-13, describes that alumina derived from a variety of pseudoboehmites have differing pore volumes and pore size distributions, despite the pseudoboehmites having similar surface areas (160˜200 m²/g).

Various synthetic methods of obtaining crystalline boehmitic alumina with high pore volume and large surface area with high thermal stability are known, e.g., from WO 00/09445 A2, WO 01/02297 A2, WO 2005/014482 A2 and WO 2016/022709 A1. For example, WO 2016/022709 Al describes boehmitic alumina with an average pore diameter of 115 to 166 Å, a bulk density of 250 to 350 kg/m³ and a pore volume of 0.8 to 1.1 m³/g, prepared by precipitation of basic aluminum salts with acidic alumina salts under controlled pH and temperature. Transition alumina produced by thermal treatment of these boehmitic aluminas and having the properties defined in the present claims are particularly suitable transition alumina for use in obtaining the porous alpha-alumina catalyst support.

Prior to heat treatment, the hydrated alumina compounds may be washed, e.g., with demineralized water, so as to reduce impurities and allow for obtaining a high purity transition alumina. For example, crystalline boehmite obtained from gibbsite by a hydrothermal process according to Chen et al., J. Solid State Chem., 265 (2018), 237 to 243, is preferably washed prior to heat treatment.

High purity transition aluminas are preferred so as to limit the content of impurities such as sodium or silicon in the catalyst support. High purity transition aluminas may be obtained by, e.g., the so-called Ziegler process and variants thereof as described in Busca, “The Surface of Transitional Aluminas: A Critical Review”, in Catalysis Today, 226 (2014), 2-13. Other processes based on the precipitation of aluminates such as sodium aluminate tend to yield transition aluminas with relatively high amounts of impurities, such as sodium.

Transition aluminas used in the present invention preferably have a total content of alkali metals, e.g., sodium and potassium, of at most 1500 ppm, more preferably at most 600 ppm and most preferably 10 ppm to 200 ppm. Various washing methods are known that allow for the reduction of the alkali metal content of the transition alumina and/or the catalyst support obtained therefrom. Washing can include washing with a base, an acid, water or other liquids.

U.S. Pat. No. 2,411,807 A describes that the sodium oxide content in alumina precipitates may be reduced by washing with a solution containing hydrofluoric acid and another acid. WO 03/086624 A1 describes carrier pretreatment with an aqueous lithium salt solution so as to remove sodium ions from the surface of a carrier. U.S. Pat. No. 3,859,426 A describes the purification of refractory oxides such as alumina and zirconia by repetitive rinsing with hot deionized water. WO 2019/039930 describes a purification method of alumina in which metal impurities were removed by extraction with an alcohol.

In a preferred embodiment, the precursor material comprises, based on inorganic solids content, at least 50 wt.-% of a transition alumina. Preferably, the precursor material comprises, based on inorganic solids content, 60 wt.-%, more preferably at least 70 wt.-% of the transition alumina, such as at least 80 wt.-% or at least 90 wt.-%, in particular 95 to 100 wt.-%.

Preferably, the transition alumina has a loose bulk density of at most 600 g/L, a pore volume of at least 0.7 mL/g, as determined by nitrogen sorption, and a median pore diameter of at least 15 nm, as determined by nitrogen sorption.

The term “loose bulk density” is understood to be the “freely settled” or “poured” density. The “loose bulk density” thus differs from the “tapped density”, where a defined mechanical tapping sequence is applied and a higher density is typically obtained. The loose bulk density may be determined by pouring the transition alumina into a graduated cylinder, suitably via a funnel, taking care not to move or vibrate the graduated cylinder. The volume and weight of the alumina are determined. The loose bulk density is determined by dividing the weight in grams by the volume in liters.

A low loose bulk density may be indicative of a high porosity and a high surface area. Preferably, the transition alumina has a loose bulk density in the range of 50 to 600 g/L, more preferably in the range of 100 to 550 g/L, most preferably 150 to 500 g/L, in particular 200 to 450 g/L.

Nitrogen sorption measurements may be performed using a Micrometrics ASAP 2420. Nitrogen porosity is determined according to DIN 66134 herein, unless stated otherwise. Preferably, the transition alumina has a pore volume of 0.7 to 2.0 mL/g, more preferably 0.7 to 1.8 mL/g, most preferably 0.8 to 1.6 mL/g, as determined by nitrogen sorption.

The term “median pore diameter” is used herein to indicate the pore diameter above which half of the total pore volume exists. Thus, the median pore diameter differentiates between two sets of pores of the same combined pore volume, with each set representing 50% of the total pore volume. The pore diameters of one set of pores are above the median pore diameter, and the pore diameters of the other set of pores are below the median pore diameter. Preferably, the transition alumina has a pore diameter of 15 to 500 nm, more preferably 20 to 450 nm, most preferably 20 to 300 nm, such as 20 to 200 nm, as determined by nitrogen sorption.

The transition alumina typically has a BET surface area in the range of 20 to 500 m²/g. The BET surface area of the transition alumina may vary over a relatively large range and may be adjusted by varying the conditions of the thermal dehydration of the hydrated aluminum compounds by which the transition alumina may be obtained. Preferably, the transition alumina has a BET surface area in the range of 20 to 200 m²/g, more preferably 50 to 150 m²/g.

Suitable transition aluminas are commercially available. In some instances, such commercially available transition aluminas are classified as “medium porosity aluminas” or, in particular, “high porosity aluminas”. Suitable transition aluminas include products of the Puralox® TH and Puralox® TM series, both from Sasol, and products of the Versal VGL series from UOP.

The transition alumina may be used in its commercially available (“unmilled”) form. Unmilled transition alumina powder typically has a D₅₀ particle diameter of 10 to 100 μm, preferably 20 to 50 μm. In addition, transition alumina may be used which has been subjected to grinding to break down the particles to a desired size. Suitably, the transition alumina may be milled in the presence of a liquid, and is preferably milled in the form of a suspension. Alternatively, grinding may be effected by dry ball-milling. Milled transition alumina powder typically has a D₅₀ particle diameter of 0.5 to 8 μm, preferably 1 to 5 μm. The particle size of transition alumina may be measured by laser diffraction particle size analyzers, such as a Malvern Mastersizer using water as a dispersing medium. The method includes dispersing the particles by ultrasonic treatment, thus breaking up secondary particles into primary particles. This sonication treatment is continued until no further change in the D₅₀ value is observed, e.g., after sonication for 3 min.

In a preferred embodiment, the transition alumina comprises at least 50 wt.-%, preferably 60 to 90 wt.-% of a transition alumina having an average particle size of 10 to 100 μm, preferably 20 to 50 μm, based on the total weight of transition alumina. Optionally, the transition alumina may comprise a transition alumina having an average particle size of 0.5 to 8 μm, preferably 1 to 5 μm, such as at most 50 wt.-%, preferably 10 to 40 wt.-%, based on the total weight of transition alumina.

In a preferred embodiment, the precursor material comprises, based on inorganic solids content, at most 30 wt.-% of an alumina hydrate. Preferably, the precursor material comprises, based on inorganic solids content, 1 to 30 wt.-% of the alumina hydrate, more preferably 1 to 25 wt.-%, most preferably 1 to 20 wt.-%, such as 3 to 18 wt.-%.

The term “alumina hydrate” is understood to relate to hydrated aluminum compounds as described above, in particular aluminum hydroxides and hydrated aluminum oxy-hydroxides. A discussion of the nomenclature of aluminas may be found in K. Wefers and C. Misra, “Oxides and Hydroxides of Aluminum”, Alcoa Laboratories, 1987. Suitable hydrated aluminum compounds include naturally occurring and synthetic compounds, such as aluminum trihydroxides like gibbsite, bayerite and nordstrandite, or aluminum oxy-monohydroxides like boehmite, pseudoboehmite and diaspore.

Preferably, the alumina hydrate comprises boehmite and/or pseudoboehmite. In a preferred embodiment, the total amount of boehmite and pseudoboehmite constitutes at least 80 wt.-%, more preferably at least 90 wt.-% and most preferably at least 95 wt.-%, such as 95 to 100 wt.-%, of the alumina hydrate. In an especially preferred embodiment, the amount of boehmite constitutes at least 80 wt.-%, more preferably at least 90 wt.-% and most preferably at least 95 wt.-%, such as 95 to 100 wt.-%, of the alumina hydrate.

Suitable alumina hydrates are commercially available and include products of the Pural® series from Sasol, preferably products of the Pural® TH and Pural® TM series, and products of the Versal® series from UOP.

Without wishing to be bound by theory, it is believed that the presence of alumina hydrate increases the mechanical stability of the support. In particular, it is believed that nano-sized, highly dispersible alumina hydrates suitable for colloidal applications, such as boehmites of the Disperal® or Dispal® series from Sasol exhibit high binding forces and can enhance the mechanical stability of the support especially efficiently. In general, using such nano-sized, highly dispersible alumina hydrates to improve mechanical stability may allow for relatively lower BET-surface areas at given calcination conditions.

Alumina hydrate may be partially or fully replaced by suitable alternative aluminum compounds while essentially retaining the mechanical stability of the support. Such suitable alternative aluminum compounds include aluminum alkoxides like aluminum ethoxide and aluminum isopropoxide, aluminum nitrate, aluminum acetate and aluminum acetylacetonate.

The precursor material may comprise a liquid. The presence, type and amount of the liquid may be chosen in accordance with the desired handling properties of the precursor material. For example, the presence of the liquid may be desirable to obtain a malleable precursor material.

The liquid is typically selected from water, in particular de-ionized water, and/or an aqueous solution comprising soluble and/or dispersible compounds selected from salts, such as ammonium acetate and ammonium carbonate; acids, such as formic acid, nitric acid, acetic acid and citric acid; bases, such as ammonia, triethylamine and methylamine; surfactants such as triethanolamine, poloxamers, fatty acid esters, and alkyl polyglucosides; submicron-sized particles, including metal oxides such as silica, titania and zirconia; clays; and/or polymer particles such as polystyrene and polyacrylates. The liquid is preferably water, most preferably de-ionized water. Typical amounts of the liquid vary in the range of from 10 to 60 wt.-%, based on the inorganic solids content of the precursor material.

The precursor material may comprise further components, such as a burnout material and/or an inorganic binder.

Suitable burnout materials include
thermally decomposable biomaterials such as acacia, sawdust, and flours, in particular ground nut shell flours, such as flours of pecan shells, cashew shells, walnut shells or filbert shells;
powdered carbonaceous compounds such as coke, or carbon powders;
milled or unmilled carbon fibers;
tableting aids such as graphite or magnesium stearate;
lubricants, such as petroleum jelly, mineral oil, or grease;
organic polymers such as
   polysaccharides, such as starch, gums, cellulose and cellulose derivatives, including substituted celluloses such as methyl cellulose, ethyl cellulose, and carboxy ethyl cellulose, and cellulose ethers;
   polyvinyl lactam polymers, such as polyvinylpyrrolidones, or vinylpyrrolidone copolymers such as vinyl pyrrolidone-vinyl acetate copolymers;
   polyolefins, like polyethylene and polypropylene;
   aromatic hydrocarbon polymers, like polystyrene;
   polycarbonates, such as poly(propylene carbonate);
   polyalkylene glycols, such as polyethylene glycol;
   lignins;
alcohols, in particular polyols such as glycol or glycerol;
fatty acid derivatives, such as esters of fatty acids, in particular esters of saturated fatty acids, such as stearate esters like methyl and ethyl stearate;
malleable organic solids such as waxes like paraffin wax, cetyl palmitate, and resins like epoxy resin and polyurethane resin; and
combinations thereof.

Burnout materials, which sometimes are also referred to as “organic binders” or “temporary binders”, may be used to maintain the porous structure of the precursor material during the “green” phase, i.e. the unfired phase, in which the mixture may be formed into shaped bodies. In general, burnout materials are essentially completely removed during calcination of the shaped bodies.

The precursor material may comprise the burnout material in amounts of 1.0 to 60 wt.-%, preferably 3 to 50 wt.-%, based on the total weight of the precursor material.

Some of the above-mentioned burnout materials, such as ground nut shell flours, constitute pore-forming materials. Such pore-forming materials may be used to improve the rate of intra-support diffusion by allowing for additional and/or wider pores in the support. The additional pore volume of wider pores can also advantageously allow for a more efficient impregnation of the support during the production of a catalyst.

Advantageously, burnout materials exhibit a low ash content. The term “ash content” is understood to relate to the incombustible component remaining after combustion of the burnout materials in air at high temperature, i.e. after calcining of the shaped bodies. The ash content is preferably below 0.1 wt.-%, relative to the weight of burnout materials.

Moreover, burnout materials preferably do not form significant amounts of volatile further combustible components, such as carbon monoxide or combustible organic compounds, upon calcining of the shaped bodies, i.e. upon thermal decomposition or combustion. An excess of volatile organic components may induce an explosive atmosphere.

Suitable inorganic binders are understood to be any of the inorganic species conventionally used in the art, e.g., silicon-containing species such as silica or silicates, including clays such as kaolinite, or metal hydroxides, metal carbonates, metal nitrates, metal acetates or metal oxides such as zirconia, titania, or alkali metal oxides.

Inorganic binders are permanent binders, which contribute to the adequate bonding of alumina particles and enhance the mechanical stability of the shaped alpha-alumina bodies.

The precursor material may comprise inorganic binders in amounts of 0.0 to 3.0 wt.-%, preferably 0.05 to 1.0 wt.-%, based on the inorganic solids content of the precursor material. In a preferred embodiment, the precursor material does not comprise an inorganic binder.

The precursor material is typically obtained by dry-mixing its components, and then optionally adding the liquid. The precursor material may be formed into shaped bodies via extrusion, tableting, granulation, casting, molding, or micro-extrusion, in particular via extrusion or tableting.

The size and shape of the shaped bodies and thus of the catalyst is selected to allow a suitable packing of the catalysts obtained from the shaped bodies in a reactor tube. The shaped catalyst bodies are preferably used in reactor tubes with a length from 6 to 14 m and an inner diameter from 20 mm to 50 mm. In general, the support is comprised of individual bodies having a maximum extension in the range of 3 to 20 mm, such as 4 to 15 mm, in particular 5 to 12 mm. The maximum extension is understood to mean the longest straight line between two points on the outer circumference of the support.

The shape of the shaped bodies is not especially limited, and may be in any technically feasible form, depending, e.g., on the forming process. For example, the support may be a solid extrudate or a hollow extrudate, such as a hollow cylinder. In another embodiment, the support may be characterized by a multilobe structure. A multilobe structure is meant to denote a cylinder structure which has a plurality of void spaces, e.g., grooves or furrows, running in the cylinder periphery along the cylinder height. Generally, the void spaces are arranged essentially equidistantly around the circumference of the cylinder.

Preferably, the support is in the shape of a solid extrudate, such as pellets or cylinders, or a hollow extrudate, such as a hollow cylinder. In a preferred embodiment, the shaped bodies are formed by extrusion, e.g., micro-extrusion. In this case, the precursor material suitably comprises a liquid, in particular water, so as to form a malleable precursor material.

In a preferred embodiment, extrusion comprises charging at least one solid component into a mixing device before the liquid is added. Preferably, a mix-muller (H-roller) or a horizontal mixer such as a Ploughshare® mixer (from Gebrüder Lödige Maschinenbau) is used for mixing. The forming of an extrudable paste of the precursor material can be monitored and controlled based on data reflecting power consumption of the mixing device.

The precursor material is typically extruded through a die. The cross-section of the die opening is adapted according to the desired geometry of the shaped body.

The extrusion die may comprise a matrix and mandrels, wherein the matrix essentially determines the circumferential shape of the shaped bodies and the mandrels essentially determine the form, size and position of passageways, if present. Suitable extrusion dies are described in, e.g., WO 2019/219892 A1.

The geometry of the shape of the shaped bodies is defined by the ideal geometry of the extrusion apparatus through which the precursor material is extruded. Generally, the geometry of the shape of the extrudate differs slightly from the ideal geometry of the extrusion apparatus, while essentially having the geometric properties described above. Absolute sizes of the shape are in general slightly lower than the sizes of the extrudate, due to high temperatures required to form alpha alumina and shrinkage upon cooling of the extrudate. The extent of the shrinkage depends on the temperatures applied during calcination and the components of the shaped bodies. Therefore, the size of the extrusion dies should be routinely adjusted in a way to account for the extrudate shrinkage during the subsequent calcination.

When the shaped body comprises multiple passageways, the axes of the passageways typically run parallel. However, the shaped bodies may be slightly bent or twisted along their z axis (height). The shape of the cross-section of the passageways may be slightly different from the envisioned perfect geometrical shapes described above. When a large amount of shaped bodies is obtained, single passageways of a small number of the shaped catalyst bodies may be closed. Usually the face sides of the shaped catalyst bodies in the xy plane are more or less uneven, rather than smooth, due to the production process. The height of the shaped bodies (length of the shaped bodies in the z direction) is usually not exactly the same for all of the shaped bodies, but rather constitutes a distribution with an average height as its arithmetic mean.

The extrudate is preferably cut into the desired length while still wet. Preferably, the extrudate is cut at an angle essentially perpendicular to its circumferential surface. In order to reduce undesirable deviations from the ideal geometry of the extrusion apparatus, the extrudate may alternatively be cut at a slanted angle of up to 30°, such as 10° or 20°, with regard to the angle perpendicular to the circumferential surface of the extrudate.

Aberrations from the ideal geometry as incurred in the extrusion process and/or the further processing of the extrudate, e.g. a cutting step, may generally also be present in the porous alpha-alumina catalyst support without essentially lessening the favorable effects of its pore structure. The skilled person understands that perfect geometrical forms are fundamentally unobtainable due to the imprecision which is inherent to all production processes to some degree.

In order to facilitate forming of the shaped bodies by extrusion, preferable burnout materials of the precursor material include processing aids such as petroleum jelly and mineral oil, grease, and/or polyalkylene glycols such as polyethylene glycol. Processing aids may be used to increase the malleability of the precursor material. The precursor material may comprise the processing aid in amounts 1.0 to 10 wt.-%, preferably 3 to 8 wt.-%, based on the inorganic solids content of the precursor material.

In another embodiment, the precursor material is formed into shaped bodies using a micro-extrusion process such as the one described in WO 2019/072597 A1.

In another embodiment, the precursor material is formed into shaped bodies via tableting. In this case, the precursor material typically does not comprise a liquid. Tableting is a process of press agglomeration. A powdered or previously agglomerated bulk material is introduced into a pressing tool having a die between two punches and compacted by uniaxial compression and shaped to give a solid compact. This operation is divided into four parts: metered introduction, compaction (elastic deformation), plastic deformation and ejection. Tableting is carried out, for example, on rotary presses or eccentric presses.

If desired, the upper punch and/or lower punch may comprise projecting pins to form internal passageways. It is also possible to provide the pressing punches with a plurality of movable pins, so that a punch can, for example, be made up of five part punches (“ring punch” having four “holes” and four pins).

The pressing force during tableting affects compaction of the bulk material. In practice, it has been found to be useful to set the lateral compressive strength of the porous alpha-alumina catalyst support in a targeted manner by selection of the appropriate pressing force and to check this by random sampling. For the purposes of the present invention, the lateral compressive strength is the force which fractures the porous alpha-alumina catalyst support located between two flat parallel plates, with the two flat parallel end faces of the catalyst support being at right angles to the flat parallel plates.

For tableting, it is often preferable to make use of tableting aids such as graphite or magnesium stearate. To improve tableting properties, a pre-granulation and/or sieving step may be used. For pre-granulation, a roll compactor, such as a Chilsonator® from Fitzpatrick, may be used. Further information regarding tableting, in particular with regard to pre-granulation, sieving, lubricants and tools, may be found in WO 2010/000720 A2.

Prior to calcining, the shaped bodies may be dried, in particular when the precursor material comprises a liquid. Suitably, drying is performed at temperatures in the range of 20 to 400° C., in particular 30 to 300° C., such as 70 to 150° C. Drying is typically performed over a period of up to 100 h, preferably 0.5 h to 30 h, more preferably 1 h to 16 h.

Drying may be performed in any atmosphere, such as in an oxygen-containing atmosphere like air, in nitrogen, or in helium, or in mixtures thereof, preferably in air. Drying is usually carried out in an oven. The type of oven is not especially limited. For example, stationary circulating air ovens, revolving cylindrical ovens or conveyor ovens may be used. Heat may be applied directly and/or indirectly.

Preferably, flue gas (vent gas) from a combustion process having a suitable temperature is used in the drying step. The flue gas may be used in diluted or non-diluted form to provide direct heating and to remove evaporated moisture and other components liberated from the shaped bodies. The flue gas is typically passed through an oven as described above. In another preferred embodiment, off-gas from a calcination process step is used for direct heating.

Drying and calcination may be carried out sequentially in separate apparatuses and may be carried out in a batch-wise or continuous process. Intermittent cooling may be applied. In another embodiment, drying and calcination are carried out in the same apparatus. In a batch process, a time-resolved temperature ramp (program) may be applied. In a continuous process, a space-resolved temperature-ramp (program) may be applied, e.g., when the shaped bodies are continuously moved through areas (zones) of different temperatures.

Preferably, measures of heat-integration as known in the art are applied in order to improve energy efficiency. For example, relatively hotter off-gas from one process step or stage can be used to heat the feed gas, apparatus or shaped bodies in another process step or stage by direct (admixing) or indirect (heat-exchanger) means. Likewise, heat integration may also be applied to cool relatively hotter off-gas streams prior to further treatment or discharge.

The shaped bodies are calcined to obtain the porous alpha-alumina catalyst support. Thus, the calcination temperature and duration are sufficient to convert at least part of the transition alumina to alpha-alumina, meaning that at least part of the metastable alumina phases of the transition alumina is converted to alpha-alumina.

The obtained porous alpha-alumina catalyst support typically comprises a high proportion of alpha-alumina, for example at least 80 wt.-%, preferably at least 90 wt.-%, more preferably at least 95 wt.-%, most preferably at least 97.5 wt.-%, based on the total weight of the support. The amount of the alpha-alumina can for example be determined via X-ray diffraction analysis.

The support of the present shaped catalyst body has a calcination history of at least 1460° C., such as 1460 to 1700° C., preferably 1475 to 1600° C., more preferably 1490 to 1550° C., most preferably 1500 to 1550° C. Preferably, calcining is performed at an absolute pressure in the range of 0.5 bar to 35 bar, in particular in the range of 0.9 to 1.1 bar, such as at atmospheric pressure (approximately 1013 mbar). Typical total heating times range from 0.5 to 100 h, preferably from 2 to 20 h.

Calcination is usually carried out in a furnace. The type of furnace is not especially limited. For example, furnaces such as stationary circulating air furnaces, revolving cylindrical furnaces or conveyor furnaces, or kilns such as rotary kilns or tunnel kilns, in particular roller hearth kilns, may be used.

Calcination may be performed in any atmosphere, such as in an oxygen-containing atmosphere like air, in nitrogen, or in helium, or in mixtures thereof. Preferably, in particular when the formed bodies contain a burnout material, calcination is at least in part or entirely carried out in an oxidizing atmosphere, such as in an oxygen-containing atmosphere like air.

As described above, burnout materials preferably do not form significant amounts of volatile further combustible components, such as carbon monoxide or combustible organic compounds, upon calcining of the shaped bodies. An explosive atmosphere may further be avoided by limiting the oxygen concentration in the atmosphere during calcination, e.g., to an oxygen concentration below the limiting oxygen concentration (LOC) with respect to the further combustible components. The LOC, also known as minimum oxygen concentration (MOC), is the limiting concentration of oxygen below which combustion is not possible.

Suitably, lean air or a gaseous recycle stream with limited oxygen content may be used along with a stream for oxygen make-up, which also compensates for gaseous purge streams. In an alternative approach, an explosive atmosphere can be avoided by limiting the rate of formation of further combustible components. The rate of formation of further combustible components may be limited by heating to the calcination temperature via a slow temperature ramp, or by heating in a step-wise manner. When heating in a step-wise manner, the temperature is suitably held for several hours at the approximate combustion temperature, then heating to temperatures of 1000° C. In a continuous calcination process, the feed rate of the shaped bodies to the calcination device, e.g., the furnace, may also be controlled so as to limit the rate of formation of further combustible components.

Depending on the nature of burnout materials and gaseous components, a waste-gas treatment may be applied in order to purify any off-gas obtained during calcination. Preferably, an acidic or alkaline scrubber, a flare or catalytic combustion, a DeNOx treatment or combinations thereof may be used for off-gas treatment.

Preferably, heating takes place in a step-wise manner. In step-wise heating, the shaped bodies may be placed on a high purity and inert refractory saggar which is moved through a furnace with multiple heating zones, e.g., 2 to 8 or 2 to 5 heating zones. The inert refractory saggar may be made of alpha-alumina or corundum, in particular alpha-alumina.

The porous alpha-alumina catalyst support typically has a BET surface in the range of 1.7 to 10 m²/g, preferably 2 to 5 m²/g, more preferably 2 to 3 m²/g. Although the BET surface of the shaped catalyst body is largely governed by the underlying support, it should be noted that deposition of silver on the support can marginally change the BET surface.

The porous alpha-alumina catalyst support typically has a total pore volume of at least 0.2 mL/g, as determined by mercury porosimetry. Mercury porosimetry may be performed using a Micrometrics AutoPore IV 9500 mercury porosimeter (140 degrees contact angle, 485 dynes/cm Hg surface tension, 60,000 psia max head pressure). The mercury porosity is determined according to DIN 66133 herein, unless stated otherwise.

The porous alpha-alumina catalyst support typically has a pore volume contained in pores with a diameter in the range of 0.1 to 1 μm of at least 40% of the total pore volume, as determined by mercury porosimetry. Preferably, the porous alpha-alumina catalyst support has a pore volume contained in pores with a diameter in the range of 0.1 to 1 μm of at least 50% of the total pore volume, more preferably at least 55% of the total pore volume, most preferably at least 60% of the total pore volume, such as at least 65% or at least 70% of the total pore volume. Typically, the porous alpha-alumina catalyst support has a pore volume contained in pores with a diameter in the range of 0.1 to 1 μm of preferably 40 to 99%, more preferably 45 to 99% most preferably 50 to 97% of the total pore volume.

The porous alpha-alumina catalyst support typically has a ratio rₚᵥ of the pore volume contained in pores with a diameter in the range of more than 1 to 10 μm to the pore volume contained in pores with a diameter in the range of 0.1 to 1 μm of at most 0.50. Preferably, the ratio of rₚᵥ is in the range of 0.0 to 0.45, more preferably 0.0 to 0.40 or 0.0 to 0.35.

The porous alpha-alumina support generally comprises at least 80 wt.-%, preferably at least 90 wt.-%, more preferably at least 95 wt.-%, most preferably at least 97.5 wt.-%, of alpha-alumina based on the total weight of the support.

In a preferred embodiment, the porous alpha-alumina support is in the form of individual shaped bodies, e.g., in a shape as described above. Preferably, the porous alpha-alumina catalyst support is in the form of individual shaped bodies having a circumferential surface, a first side surface, a second side surface and at least one internal passageway extending from the first side surface to the second side surface.

Preferably, the quotient of the geometric surface of the catalyst support SA_{geo} over the geometric volume of the catalyst support V_{geo} (SA_{geo}/V_{geo}) is at least 1.1 mm⁻, and at most 10 mm⁻¹. Preferably, the quotient of SA_{geo} over V_{geo} is in the range of 1.15 mm⁻¹ to 5.0 mm⁻¹, more preferably in the range of 1.2 mm⁻¹ to 2.0 mm⁻¹. The geometric surface area SA_{geo} and the geometric volume V_{geo} are derived from the external, macroscopic dimensions of the porous alpha-alumina catalyst support taking into account the cross-sectional area, the height and, where applicable, the number of internal passageways. In other words, the geometric volume V_{geo} of the catalyst support is the volume of a solid structure having the same outer dimensions, minus the volume occupied by passageways. Likewise, the geometric surface area SA_{geo} is made up of the circumferential surface, the first and second face side surface and, where applicable, the surface defining the passageways. The first and second face side surface, respectively, is the surface area enclosed by the circumferential line of the face side, minus the cross-sectional areas of the passageways. The surface defining the passageways is the surface area lining the passageways.

A quotient of SA_{geo} over V_{geo} in the preferred range makes it possible for a better contact of the reaction gases with the catalyst surface to be obtained, which favors the conversion of the reactants and limits the inner diffusion phenomena, with a resulting increase in reaction selectivity.

The porous alpha-alumina support preferably does not have wash-coat particles or a wash-coat layer on its surface, so as to fully maintain the porosity of the uncoated support.

The porous alpha-alumina catalyst support may comprise impurities, such as sodium, potassium, magnesium, calcium, silicon, iron and/or zirconium. Such impurities may be introduced by components of the precursor material, in particular inorganic binders. In one embodiment, the porous alpha-alumina catalyst support comprises
a total amount of 10 to 1,500 ppmw of sodium and potassium;
10 to 2,000 ppmw of calcium;
10 to 1,000 ppmw of magnesium;
10 to 10,000 ppmw of silicon; and/or
10 to 1,000 ppmw of iron;
   relative to the total weight of the support.

A low content of sodium is preferred in order to prevent segregation of the supported metal and to prevent alteration of the supported component.

The shaped catalyst body comprises silver, typically 8 to 40 wt.-% of silver, preferably 15 to 35 wt.-% of silver, more preferably 25 to 35 wt.-% of silver, relative to the total weight of the shaped catalyst body. A silver content in this range allows for a favorable balance between turnover induced by each shaped catalyst body and cost-efficiency of preparing the shaped catalyst body.

The shaped catalyst body comprises a rhenium promoter. A promoter denotes a component that provides an improvement in one or more of the catalytic properties of the catalyst when compared to a catalyst not containing said component. A promoter can be any of those species known in the art that function to improve the catalytic properties of the silver catalyst. Examples of catalytic properties include operability (resistance to runaway), selectivity, activity, turnover and catalyst longevity.

The shaped catalyst body typically comprises 200 to 2000 ppm of rhenium, preferably 400 to 1800 ppm of rhenium, more preferably 600 to 1600 ppm, most preferably 800 to 1400 ppm of rhenium, relative to the total weight of the shaped catalyst body.

Rhenium (Re) is a particularly efficacious transition metal promoter for ethylene epoxidation high selectivity catalysts. The rhenium component in the shaped catalyst body can be in any suitable form, but is more typically one or more rhenium-containing compounds (e.g., a rhenium oxide) or complexes.

The shaped catalyst body may further comprise a promoting amount of a transition metal or a mixture of two or more additional transition metals besides rhenium. Suitable additional transition metals can include the elements from Groups IIIB (scandium group), IVB (titanium group), VB (vanadium group), VIB (chromium group), VIIB (manganese group), VIII B (iron, cobalt, nickel groups), IB (copper group), and IIB (zinc group) of the Periodic Table of the Elements, as well as combinations thereof. More typically, the additional transition metal is an early transition metal, i.e., from Groups IIIB, IVB, VB or VIB, such as, for example, hafnium, yttrium, molybdenum, tungsten, chromium, titanium, zirconium, vanadium, tantalum, niobium, or a combination thereof. The most preferable additional transition metal promoters are tungsten, molybdenum and/or manganese. In one embodiment, the additional transition metal promoter(s) is (are) present in a total amount from 200 ppm to 1000 ppm, typically 300 ppm to 800 ppm, most typically from 400 ppm to 700 ppm, expressed in terms of metal(s) relative to the total weight of the shaped catalyst body.

In some embodiments, the shaped catalyst body may include a promoting amount of an alkali metal or a mixture of two or more alkali metals. Suitable alkali metal promoters include, for example, lithium, sodium, potassium, rubidium, cesium or combinations thereof.

The amount of potassium, if present, will typically range from 50 ppm to 500 ppm, more typically from 75 ppm to 400 ppm, most typically from 100 ppm to 300 ppm, expressed in terms of potassium relative to the total weight of the shaped catalyst body.

The amount of cesium, if present, will typically range from 300 ppm to 2000 ppm, more typically from 500 ppm to 1500 ppm, most typically from 700 ppm to 1200 ppm, expressed in terms of cesium relative to the total weight of the shaped catalyst body.

The amount of lithium, if present, will typically range from 50 ppm to 1000 ppm, more typically from 100 ppm to 800 ppm, most typically from 200 ppm to 600 ppm, expressed in terms of lithium relative to the total weight of the shaped catalyst body.

The amount of sodium, if present, will typically range from 20 ppm to 500 ppm, more typically from 50 ppm to 300 ppm, most typically from 75 ppm to 200 ppm, expressed in terms of sodium relative to the total weight of the shaped catalyst body.

The amount of alkali metal is determined by the amount of alkali metal contributed by the porous alpha-alumina catalyst support and the amount of alkali metal contributed by the impregnation solution described below.

Combinations of heavy alkali metals like cesium (Cs) or rubidium (Rb) with light alkali metals like lithium (Li), sodium (Na) and potassium (K) are particularly preferred. Combinations of cesium (Cs), potassium (K), lithium (Li) and optionally sodium (Na) are most preferred.

The shaped catalyst body may also include a Group IIA alkaline earth metal or a mixture of two or more Group IIA alkaline earth metals. Suitable alkaline earth metal promoters include, for example, beryllium, magnesium, calcium, strontium, and barium or combinations thereof. The amounts of alkaline earth metal promoters can be used in amounts similar to those used for the alkali or transition metal promoters.

The shaped catalyst body may also include a promoting amount of a main group element or a mixture of two or more main group elements. Suitable main group elements include any of the elements in Groups IIIA (boron group) to VIIA (halogen group) of the Periodic Table of the Elements. For example, the shaped catalyst body can include a promoting amount of sulfur, phosphorus, boron, halogen (e.g., fluorine), gallium, or a combination thereof.

The shaped catalyst body may also include a promoting amount of a rare earth metal or a mixture of two or more rare earth metals. The rare earth metals include any of the elements having an atomic number of 57-103. Some examples of these elements include lanthanum (La), cerium (Ce), and samarium (Sm). The amount of rare earth metal promoters can be used in amounts similar to those used for the transition metal promoters.

In a preferred embodiment, the shaped catalyst body includes a promoting amount of a promoter selected from cesium, potassium, lithium, tungsten and/or sulfur.

The shaped catalyst body of the invention is obtainable by depositing silver and rhenium on a porous alpha-alumina support having a BET surface area of 1.7 to 10 m²/g and a calcination history of at least 1460° C. In particular, the shaped catalyst body of the invention is obtainable by depositing silver and rhenium on a porous alpha-alumina support as described above.

Specifically, the shaped catalyst body of the invention is obtainable by successive steps of impregnating the porous alpha-alumina support with an impregnation solution and subjecting the impregnated porous alpha-alumina support to a heat treatment or a series of successive steps of impregnating and heat treatment, wherein at least one of the impregnation solutions used contains a silver compound and at least one of the impregnation solutions used contains a rhenium compound.

The deposition of silver and rhenium may typically be achieved by
1) impregnating the porous alpha-alumina catalyst support as described above with an impregnation solution, preferably under reduced pressure; and optionally subjecting the impregnated porous alumina support to drying; and
2) subjecting the impregnated porous alpha-alumina support to a heat treatment;
   wherein steps 1) and 2) are optionally repeated.

In order to obtain a shaped catalyst body having high silver contents, steps 1) and 2) can be repeated once or several times. In this case it is understood that the intermediate product obtained after the first (or subsequent up to the last but one) impregnation/calcination cycle comprises a part of the total amount of target Ag and/or Re concentrations. The intermediate product is then again impregnated with the silver impregnation solution and calcined to yield the target Ag and/or Re concentrations.

When steps 1) and 2) are repeated, the composition of the impregnation solution may be different in each iteration. At least one impregnation solution comprises at least one silver compound, and at least one impregnation solution comprises at least one rhenium compound, wherein the impregnation solution may comprise both a silver compound and a rhenium compound. For example, the porous alpha-alumina support may be impregnated twice with an impregnation solution comprising a silver compound (a “silver impregnation solution”), being subjected to a heat treatment after each impregnation step, and finally impregnated with an impregnation solution comprising both a silver compound and a rhenium compound, being subjected to a heat treatment thereafter. When steps 1) and 2) are not repeated, the impregnation solution necessarily comprises at least one silver compound and at least one rhenium compound.

Any silver impregnation solution suitable for impregnating a refractory support known in the art can be used. Silver impregnation solutions typically contain a silver carboxylate, such as silver oxalate, or a combination of a silver carboxylate and oxalic acid, in the presence of an aminic complexing agent like a C₁-C₁₀-alkylenediamine, in particular ethylenediamine. Suitable impregnation solutions are described in EP 0 716 884 A2, EP 1 115 486 A1, EP 1 613 428 A1, U.S. Pat. No. 4,731,350 A, WO 2004/094055 A2, WO 2009/029419 A1, WO 2015/095508 A1, U.S. Pat. Nos. 4,356,312 A, 5,187,140 A, 4,908,343 A, 5,504,053 A and WO 2014/105770 A1.

During heat treatment, liquid components of the silver impregnation solution evaporate, causing a silver compound comprising silver ions to precipitate from the solution and be deposited onto the porous support. At least part of the deposited silver ions is subsequently converted to metallic silver upon further heating. Preferably, at least 70 mol-% of the silver compounds, preferably at least 90 mol-%, more preferably at least 95 mol-% and most preferably at least 99.5 mol-% or at least 99.9 mol-%, i.e. essentially all of the silver ions, based on the total molar amount of silver in the impregnated porous alpha-alumina support, respectively. The amount of the silver ions converted to metallic silver can for example be determined via X-ray diffraction (XRD) patterns.

The heat treatment may also be referred to as a calcination process. Any calcination processes known in the art for this purpose can be used. Suitable examples of calcination processes are described in U.S. Pat. Nos. 5,504,052 A, 5,646,087 A, 7,553,795 A, 8,378,129 A, 8,546,297 A, US 2014/0187417 A1, EP 1 893 331 A1 or WO 2012/140614 A1. Heat treatment can be carried out in a pass-through mode or with at least partial recycling of the calcination gas.

Heat treatment is usually carried out in a furnace. The type of furnace is not especially limited. For example, stationary circulating air furnaces, revolving cylindrical furnaces or conveyor furnaces may be used. In one embodiment, heat treatment constitutes directing a heated gas stream over the impregnated bodies. The duration of the heat treatment is generally in the range of 5 min to 20 h, preferably 5 min to 30 min.

The temperature of the heat treatment is generally in the range of 200 to 800° C., preferably 210 to 650° C., more preferably 220 to 500° C., most preferably 220 to 350° C. Preferably, the heating rate in the temperature range of 40 to 200° C. is at least 20 K/min, more preferably at least 25 K/min, such as at least 30 K/min. A high heating rate may be achieved by directing a heated gas over the impregnated refractory support or the impregnated intermediate catalyst at a high gas flow.

A suitable flow rate for the first gas and/or the second gas may be in the range of, e.g., 1 to 1,000 Nm³/h, 10 to 1,000 Nm³/h, 15 to 500 Nm³/h or 20 to 300 Nm³/h per kg of impregnated bodies. In a continuous process, the term “kg of impregnated bodies” is understood to mean the amount of impregnated bodies (in kg/h) multiplied by the time (in hours) that the gas stream is directed over the impregnated bodies. It has been found that when the gas stream is directed over higher amounts of impregnated bodies, e.g., 15 to 150 kg of impregnated bodies, the flow rate may be chosen in the lower part of the above-described ranges, while achieving the desired effect.

Determining the temperature of the heated impregnated bodies directly may pose practical difficulties. Hence, when a heated gas is directed over the impregnated bodies during heat treatment, the temperature of the heated impregnated bodies is considered to be the temperature of the gas immediately after the gas has passed over the impregnated bodies. In a practical embodiment, the impregnated bodies are placed on a suitable surface, such as a wire mesh or perforated calcination belt, and the temperature of the gas is measured by one or more thermocouples positioned adjacent to the opposite side of the impregnated bodies which first comes into contact with the gas. The thermocouples are suitably placed close to the impregnated bodies, e.g., at a distance of 1 to 30 mm, such as 1 to 3 mm or 15 to 20 mm from the impregnated bodies.

The use of a plurality of thermocouples can improve the accuracy of the temperature measurement. Where several thermocouples are used, these may be evenly spaced across the area on which the impregnated bodies rest on the wire mesh, or the breadth of the perforated calcination belt. The average value is considered to be the temperature of the gas immediately after the gas has passed over the impregnated bodies. To heat the impregnated bodies to the temperatures as described above, the gas typically has a temperature of 220 to 800° C., more preferably 230 to 550° C., most preferably 240 to 350° C.

Preferably, heating takes place in a step-wise manner. In step-wise heating, the impregnated bodies are placed on a moving belt that moves through a furnace with multiple heating zones, e.g., 2 to 8 or 2 to 5 heating zones. Heat treatment is preferably performed in an inert atmosphere, such as nitrogen, helium, or mixtures thereof, in particular in nitrogen.

The invention further relates to a process for producing ethylene oxide by gas-phase oxidation of ethylene, comprising reacting ethylene and oxygen in the presence of a shaped catalyst body as described above.

It is understood that all embodiments of the shaped catalyst body also apply to the process for producing ethylene oxide in the presence of the shaped catalyst body, where applicable.

The epoxidation can be carried out by all processes known to those skilled in the art. It is possible to use all reactors which can be used in the ethylene oxide production processes of the prior art; for example externally cooled shell-and-tube reactors (cf. Ullmann's Encyclopedia of Industrial Chemistry, 5th edition, vol. A-10, pp. 117-135, 123-125, VCH-Verlagsgesellschaft, Weinheim 1987) or reactors having a loose catalyst bed and cooling tubes, for example the reactors described in DE 34 14 717 A1, EP 0 082 609 A1 and EP 0 339 748 A2.

The epoxidation is preferably carried out in at least one tube reactor, preferably in a shell-and-tube reactor. On a commercial scale, ethylene epoxidation is preferably carried out in a multi-tube reactor that contains several thousand tubes. The catalyst is filled into the tubes, which are placed in a shell that is filled with a coolant. In commercial applications, the internal tube diameter is typically in the range of 20 to 40 mm (see, e.g., U.S. Pat. No. 4,921,681 A) or more than 40 mm (see, e.g., WO 2006/102189 A1).

To prepare ethylene oxide from ethylene and oxygen, it is possible to carry out the reaction under conventional reaction conditions as described, for example, in DE 25 21 906 A, EP 0 014 457 A2, DE 23 00 512 A1, EP 0 172 565 A2, DE 24 54 972 A1, EP 0 357 293 A1, EP 0 266 015 A1, EP 0 085 237 A1, EP 0 082 609 A1 and EP 0 339 748 A2. Inert gases such as nitrogen or gases which are inert under the reaction conditions, e.g. steam, methane, and also optionally reaction moderators, for example halogenated hydrocarbons such as ethyl chloride, vinyl chloride or 1,2-dichloroethane can additionally be mixed into the reaction gas comprising ethylene and molecular oxygen.

The oxygen content of the reaction gas is advantageously in a range in which no explosive gas mixtures are present. A suitable composition of the reaction gas for preparing ethylene oxide can, for example, comprise an amount of ethylene in the range from 10 to 80% by volume, preferably from 20 to 60% by volume, more preferably from 25 to 50% by volume and particularly preferably in the range from 25 to 40% by volume, based on the total volume of the reaction gas. The oxygen content of the reaction gas is advantageously in the range of not more than 10% by volume, preferably not more than 9% by volume, more preferably not more than 8% by volume and very particularly preferably not more than 7.5% by volume, based on the total volume of the reaction gas.

The reaction gas preferably comprises a chlorine-comprising reaction moderator such as ethyl chloride, vinyl chloride or 1,2-dichloroethane in an amount of from 0 to 15 ppm by weight, preferably in an amount of from 0.1 to 8 ppm by weight, based on the total weight of the reaction gas. The remainder of the reaction gas generally comprises hydrocarbons such as methane and also inert gases such as nitrogen. In addition, other materials such as steam, carbon dioxide or noble gases can also be comprised in the reaction gas.

The concentration of carbon dioxide in the feed (i.e. the gas mixture fed to the reactor) typically depends on the catalyst selectivity and the efficiency of the carbon dioxide removal equipment. Carbon dioxide concentration in the feed is preferably at most 3 vol.-%, more preferably less than 2 vol.-%, most preferably less than 1 vol.-%, relative to the total volume of the feed. An example of carbon dioxide removal equipment is provided in U.S. Pat. No. 6,452,027 B1.

The above-described constituents of the reaction mixture may optionally each have small amounts of impurities. Ethylene can, for example, be used in any degree of purity suitable for the gas-phase oxidation according to the invention. Suitable degrees of purity include, but are not limited to, “polymer-grade” ethylene, which typically has a purity of at least 99%, and “chemical-grade” ethylene which typically has a purity of less than 95%. The impurities typically comprise, in particular, ethane, propane and/or propene.

The reaction or oxidation of ethylene to ethylene oxide is usually carried out at elevated catalyst temperatures. Preference is given to catalyst temperatures in the range of 150 to 350° C., more preferably 180 to 300° C., particularly preferably 190 to 280° C. and especially preferably 200 to 280° C. The present invention therefore also provides a process as described above in which the oxidation is carried out at a catalyst temperature in the range 180 to 300° C., preferably 200 to 280° C. Catalyst temperature can be determined by thermocouples located inside the catalyst bed. As used herein, the catalyst temperature or the temperature of the catalyst bed is deemed to be the weight average temperature of the catalyst particles.

The reaction according to the invention (oxidation) is preferably carried out at pressures in the range of 5 to 30 bar. All pressures herein are absolute pressures, unless noted otherwise. The oxidation is more preferably carried out at a pressure in the range of 5 to 25 bar, such as 10 bar to 24 bar and in particular 14 bar to 23 bar. The present invention therefore also provides a process as described above in which the oxidation is carried out at a pressure in the range of 14 bar to 23 bar.

It has been found that the physical characteristics of the shaped catalyst body, especially the BET surface area and the pore size distribution have a significant positive impact on the catalyst selectivity. This effect is especially distinguished when the catalyst is operated at very high work rates, i.e., high levels of olefin oxide production.

The process according to the invention is preferably carried out under conditions conducive to obtain a reaction mixture containing at least 2.3 vol.-% of ethylene oxide. In other words, the ethylene oxide outlet concentration (ethylene oxide concentration at the reactor outlet) is preferably at least 2.3 vol.-%. The ethylene oxide outlet concentration is more preferably in the range of 2.5 to 4.0 vol.-%, most preferably in the range of 2.7 to 3.5 vol.-%.

The oxidation is preferably carried out in a continuous process. If the reaction is carried out continuously, the GHSV (gas hourly space velocity) is, depending on the type of reactor chosen, for example on the size/cross-sectional area of the reactor, the shape and size of the catalyst, preferably in the range from 800 to 10,000/h, preferably in the range from 2,000 to 8,000/h, more preferably in the range from 2,500 to 6,000/h, most preferably in the range from 4,500 to 5,500/h, where the values indicated are based on the volume of the catalyst.

According to a further embodiment, the present invention is also directed to a process for preparing ethylene oxide (EO) by gas-phase oxidation of ethylene by means of oxygen as disclosed above, wherein the EO-space-time-yield measured is greater than 180 kg_{EO}/(m³_{cat}h), preferably to an EO-space-time-yield of greater than 200 kg_{EO}/(m³_{cat}h), such as greater than 250 kg_{EO}/(m³_{cat}h), greater than 280 kg_{EO}/(m³_{cat}h), or greater than 300 kg_{EO}/(m³_{cat}h). Preferably the EO-space-time-yield measured is less than 500 kg_{EO}/(m³_{cat}h), more preferably the EO-space-time-yield is less than 350 kg_{EO}/(m³_{cat}h).

The preparation of ethylene oxide from ethylene and oxygen can advantageously be carried out in a recycle process. After each pass, the newly formed ethylene oxide and the by-products formed in the reaction are removed from the product gas stream. The remaining gas stream is supplemented with the required amounts of ethylene, oxygen and reaction moderators and reintroduced into the reactor. The separation of the ethylene oxide from the product gas stream and its work-up can be carried out by customary methods of the prior art (cf. Ullmann's Encyclopedia of Industrial Chemistry, 5th edition, vol. A-10, pp. 117-135, 123-125, VCH-Verlagsgesellschaft, Weinheim 1987).

The invention is described in more detail by the subsequent examples.

Method 1: Nitrogen Sorption

Nitrogen sorption measurements were performed using a Micrometrics ASAP 2420. Nitrogen porosity was determined in accordance with DIN 66134.

Method 2: Mercury Porosimetry

Mercury porosimetry was performed using a Micrometrics AutoPore IV 9500 mercury porosimeter (140 degrees contact angle, 485 dynes/cm Hg surface tension, 60,000 psia max head pressure). Mercury porosity was determined in accordance with DIN 66133.

Method 3: Loose Bulk Density

The loose bulk density was determined by pouring the transition alumina or alumina hydrate into a graduated cylinder via a funnel, taking care not to move or vibrate the graduated cylinder. The volume and weight of the transition alumina or alumina hydrate were determined. The loose bulk density was determined by dividing the volume in milliliters by the weight in grams.

Method 4: BET Surface Area

The BET surface area was determined in accordance with DIN ISO 9277.

Method 5:

Analysis of the Total Amount of Ca—, Mg—, Si—, Fe—, K—, and Na-Contents in Alpha-Alumina Supports

5A. Sample Preparation for Measurement of Ca, Mg, Si and Fe

About 100 to 200 mg (at an error margin of ±0.1 mg) of a carrier sample were weighed into a platinum crucible. 1.0 g of lithium metaborate (LiBO₂) was added. The mixture was melted in an automated fusion apparatus with a temperature ramp up to max. 1150° C.

After cooling down, the melt was dissolved in deionized water by careful heating. Subsequently, 10 mL of semi-concentrated hydrochloric acid (concentrated HCl diluted with deionized water, volume ratio 1:1, corresponds to about 6 M) was added. Finally, the solution was filled up to a volume of 100 mL with deionized water.

5B. Measurement of Ca, Mg, Si and Fe

The amounts of Ca, Mg, Si and Fe were determined from the solution described under item 5A by Inductively Coupled Plasma-Optical Emission Spectroscopy (ICP-OES) using an ICP-OES Varian Vista Pro.
Parameters:
Wavelengths [nm]: Ca 317.933
Mg 285.213
Si 251.611
Fe 238.204
Integration time: 10 s
Nebulizer: Conikal 3 ml
Nebulizer pressure: 270 kPa
Pump rate: 30 rpm
Calibration: external (matrix-matched standards)

5C. Sample Preparation for Measurement of K and Na

About 100 to 200 mg (at an error margin of ±0.1 mg) of a carrier sample were weighed into a platinum dish. 10 mL of a mixture of aqueous concentrated H₂SO₄ (95 to 98%) and deionized water (volume ratio 1:4), and 10 mL of aqueous hydrofluoric acid (40%) were added. The platinum dish was placed on a sand bath and boiled down to dryness. After cooling down the platinum dish, the residue was dissolved in deionized water by careful heating. Subsequently, 5 mL of semi-concentrated hydrochloric acid (concentrated HCl diluted with deionized water, volume ratio 1:1, corresponds to about 6 M) were added. Finally, the solution was filled up to a volume of 50 mL with deionized water.

5D. Measurement of K and Na

The amounts of K and Na were determined from the solution described under item 5C by Flame Atomic Absorption Spectroscopy (F-AAS) using an F-AAS Shimadzu AA-7000.
Parameters:
Wavelengths [nm]: K 766.5 Na 589.0
Gas: Air/acetylene
Slit width: 0.7 nm (K)/0.2 nm (Na)
Nebulizer pressure: 270 kPa
Calibration: external (matrix-matched standards)

Method 6: Ethylene Oxide Decomposition Test

An ethylene oxide decomposition reaction was conducted in a vertically-placed test reactor constructed from stainless steel with an inner diameter of 6 mm and a length of 200 cm. The reactor was heated electrically at a temperature in the range of 150 to 300° C. and at an inlet pressure of 15 bar. The reactor was filled to a specified height with inert steatite balls (e.g., 1.0-1.6 mm), packed with a specified amount of catalyst support, and then optionally again packed with additional inert steatite balls (1.0 to 1.6 mm). Prior to filling the catalyst into the reactor, the catalyst support bodies were gently broken into pieces of a specified particle size. The inlet gas was introduced to the top of the reactor in a “once-through” operation mode.

The catalyst support was initially heated under nitrogen flow to a temperature of 200 to 300° C. for 1 to 12 hours to remove residual moisture. Subsequently, the inlet gas was set to 2.0 to 3.5 vol.-% ethylene oxide, 3 to 5 vol.-% oxygen, 1 to 3 vol.-% carbon dioxide, 25 to 35 vol.-% ethylene, 50 to 65 vol.-% methane, 0.5 to 2.5 vol.-% steam and a mixture of ethylene chloride (EC), vinyl chloride (VC), and methyl chloride (MC) in the range of 1 to 10 parts per million by volume (ppmv).

Products were identified using online mass spectrometry.

Ethylene oxide (EO) conversion was determined as

(EOinlet−EOoutlet)/EOinlet*100

EOᵢₙₗₑₜ is the concentration of ethylene oxide at the reactor inlet, measured in vol-%.

EOₒᵤₜₗₑₜ is the concentration of ethylene oxide at the reactor outlet, measured in vol-%.

### EXAMPLE 1

### Preparation of Porous Alpha-Alumina Catalyst Supports

The properties of the transition aluminas and alumina hydrates used to obtain porous alpha-alumina catalyst supports are shown in Table 1. The transition aluminas and alumina hydrates were obtained from Sasol.

TABLE 1
Transition Aluminas

| | Bulk | Pore Volume | Median Pore |
|---|---|---|---|
| | Density [g/L] | [mL/g] * | Diameter [nm] * |
| Puralox TH 200/70 | 300 | 1.23 | 37.4 |
| Puralox SCFa 140 | 650 | 0.57 | 10.0 |
| Puralox TM 100/150 UF | 150 | 0.88 | 18.4 |

Alumina Hydrates

| | Bulk | Pore Volume | Median Pore |
|---|---|---|---|
| | Density [g/L] | [mL/g] | Diameter [nm] |
| Pural SB1 | 680 | 0.55 | 8.4 |
| Pural TH 200 | 340 | 1.20 | 37.6 |

* determined by nitrogen sorption

Transition aluminas and alumina hydrates, as specified in Table 1, were mixed to obtain a powder mixture. Colloidal silica (Ludox® AS 40, Grace & Co.) and petroleum jelly (Vaseline®, Unilever) were added to the powder mixture. Water was then added to obtain a malleable precursor material. The amounts of all components are shown in Table 2.

TABLE 2

| | Transition | Alumina | | Processing | |
|---|---|---|---|---|---|
| Support | Alumina | Hydrate | Binder | Aid | Liquid |
| A | Puralox TH 200/70 | Pural TH | Silica | Petroleum | Water |
| | 320 g | 200 17 g | Sol | Jelly | 439 g |
| | Puralox TM 100/150 UF | | 1.5 g | 23.8 g | |
| | 138 g | | | | |
| B * | Puralox SCFa 140 | Pural SB1 | Silica | Petroleum | Water |
| | 320 g | 17 g | Sol | Jelly | 364 g |
| | Puralox TM 100/150 UF | | 1.5 g | 23.9 g | |
| | 138 g | | | | |

* comparative example

The malleable precursor material was mixed to homogeneity via a mix-muller and subsequently extruded using a ram extruder to form shaped bodies. The shaped bodies were in the form of hollow cylinders having an outer diameter of about 10 mm and an inner diameter of about 5 mm. The extrudates were dried at 110° C. for approximately 16 h, followed by heat treatment in a muffle furnace at 600° C. for 2 h. Subsequently, the extrudates were heat treated at 1,500° C. for 4 h (to obtain support A), and at 1425° C. for 4 h, respectively (to obtain support B). Heat treatment was performed in an atmosphere of air

Table 3 shows the Si—, Ca—Mg—, Na—, K— and Fe-contents in alumina supports A and B, relative to the total weight of the support.

TABLE 3

| | Si_{Al2O3} | Na_{Al2O3} | K_{Al2O3} | Fe_{Al2O3} | Ca_{Al2O3} | Mg_{Al2O3} |
|---|---|---|---|---|---|---|
| Support | [ppmw] | [ppmw] | [ppmw] | [ppmw] | [ppmw] | [ppmw] |
| A | 700 | 40 | <30 | 300 | <100 | <100 |
| B * | 800 | 50 | <30 | 100 | 100 | <100 |

* comparative example

Support A had a BET surface area of 4.8 m²/g. Support B had a BET surface area of 4.4 m²/g.

### EXAMPLE 2

### Preparation of Catalysts

Shaped catalyst bodies were prepared by impregnating supports A and B with a silver impregnation solution. The catalyst compositions and BET surface areas are shown in Table 4 below. Silver contents are provided in percent, relative to the total weight of the catalyst. Dopant values are provided in parts per million, relative to the total weight of the catalyst.

TABLE 4

| | | | | | | | | | BET |
|---|---|---|---|---|---|---|---|---|---|
| | | | | | | | | | Surface |
| | | Ag_{CAT} ** | Li_{CAT} | S_{CAT} | W_{CAT} | Cs_{CAT} | Re_{CAT} | K_{ADD} *** | Area |
| Catalyst | Support | [wt-%] | [ppm] | [ppm] | [ppm] | [ppm] | [ppm] | [ppm] | [m²/g] |
| 1 | A | 10.0 | 282 | 21 | 342 | 570 | 720 | 31 | 4.9 |
| 2 * | B | 10.0 | 282 | 21 | 342 | 570 | 720 | 31 | 5.1 |

* comparative example
** Ag and all promoter values are calculated values
*** K_{ADD} is understood to mean the amount of potassium added during impregnation and does not include the amount of potassium comprised in the alumina support prior to impregnation

### 2.1 Production of a Silver Complex Solution

A silver complex solution was prepared according to Production Example 1 of WO 2019/154863 A1. The silver complex solution had a density of 1.529 g/mL, a silver content of 29.3 wt-% and a potassium content of 90 ppm.

### 2.2 Production of Catalyst 1

94.9 g of support A as described in Example 1 were placed into a 1 L glass flask. The flask was attached to a rotary evaporator which was set under vacuum pressure of 80 mbar. The rotary evaporator system was set in rotation of 30 rpm.

36.03 g of the silver complex solution prepared according to production example 2.1 was mixed with 1.045 g of promoter solution I, 1.203 g of promoter solution II, and 2.054 g of promoter solution III. Promoter solution I was made from dissolving lithium nitrate (FMC, 99.3%) and ammonium sulfate (Merck, 99.4%) in deionized water to achieve a Li content of 2.85 wt.-% and S content of 0.21 wt.-%. Promoter solution II was made from dissolving tungstic acid (HC Starck, 99.99%) in deionized water and cesium hydroxide in water (HC Starck, 50.42%) to achieve a Cs content of 5.0 wt.-% and W content of 3.0 wt.-%. Promoter solution III was made from dissolving ammonium perrhenate (Engelhard, 99.4%) in deionized water to achieve a Re content of 3.7 wt.-%.

The combined impregnation solution containing the silver complex solution and promoter solutions I, II, and III was stirred for 5 min. The combined impregnation solution was added onto the support A over 15 min under a vacuum of 80 mbar. After addition of the combined impregnation solution, the rotary evaporator system was continued to rotate under vacuum for another 15 min. The impregnated support was then left in the apparatus at room temperature and atmospheric pressure for 1 h and mixed gently every 15 min. The impregnated material was placed on a net forming 1 to 2 layers (about 100 too 200 g per calcination run). The net was subjected to 23 m³/h nitrogen flow (oxygen content: <20 ppm). The impregnated material was heated up to a temperature of 290° C. at a heating rate of about 30 K/min and then held at 290° C. for 8 min.

### 2.3 Production of Comparative Catalyst 2

Catalyst 2 was prepared in the same manner as Catalyst 1, except that support B was used instead of support A.

### 2.4 Catalyst Testing

An epoxidation reaction was conducted in a vertically-placed test reactor constructed from stainless steel with an inner diameter of 6 mm and a length of 2.2 m. The reactor was heated using hot oil contained in a heating mantel at a specified temperature. All reactor temperatures below refer to the temperature of the hot oil. The reactor was filled to a height of 212 mm with inert steatite balls (1.0-1.6 mm), packed with 26.4 g of catalyst, and then again packed with an additional 707 mm inert steatite balls (1.0 to 1.6 mm). Prior to filling the catalyst into the reactor, the catalyst shaped bodies were gently broken into pieces of 0.3 to 0.7 mm. The inlet gas was introduced to the top of the reactor in a “once-through” operation mode.

The catalyst was conditioned in the inlet gas consisted of 20 vol.-% ethylene, 4 vol.-% oxygen, 1 vol.-% carbon dioxide, and ethylene chloride (EC) moderation of 2.5 parts per million by volume (ppmv), with methane used as a balance at the total gas flow rate of 152.7 NL/h, at a pressure of about 15 bar. During the conditioning phase, the catalyst was heated up from 210° C. to 250° C. at a heating ramp of 4° C./h. Then the catalyst was held at 250° C. for 8 hours. Then, within an hour, the temperature was reduced to 240° C. and held for 4 hours. Then, within an hour, the temperature was reduced to 230° C. and held for 2 hours. Then, within two hours, the temperature was reduced to 220° C. and held for 17 hours.

After the conditioning phase, the inlet gas composition was gradually changed to 35 vol.-% ethylene, 7 vol.-% oxygen, 1 vol.-% carbon dioxide with methane used as a balance and a total gas flow rate of 147.9 NL/h. The temperature was adjusted to achieve an ethylene oxide (EO) concentration in the outlet gas of 1.95 vol-%. The EC concentration was varied in the range of 2.2 to 3.5 ppmv to optimize the catalyst selectivity. Results of the catalyst tests at an optimal EC concentration are summarized in Table 5.

TABLE 5
Test Reaction Results

| | | Support | | | Reactor |
|---|---|---|---|---|---|
| | | Calcination | Catalyst | EO- | Temper- |
| | | Temperature | Amount | Selectivity | ature |
| Catalyst | Support | [° C.] | [g] | [%] | [° C.] |
| 1 | A | 1,500 | 26.4 | 78.5 | 210.5 |
| 2 * | B | 1,425 | 26.4 | 71.6 ** | 220.5-217.0 |

* comparative example
** catalyst 2 exhibited an initial selectivity of 71.6%, which quickly fell to 51.8%

It is evident that catalyst 1, based on support A, shows a much higher selectivity than catalyst 2, based on support B. Catalyst 1 also shows a higher activity than catalyst 2, as is evident from the lower reactor temperature. Moreover, catalyst 2 did not allow for a stable operation at the final inlet gas composition.

1.-15. (canceled)

16. A shaped catalyst body for producing ethylene oxide by gas-phase oxidation of ethylene, having a BET surface area in the range of 2 to 20 m²/g and comprising silver and a rhenium promotor deposited on a porous alpha-alumina catalyst support, characterized in that the support has a calcination history of at least 1460° C.

17. The shaped catalyst body according to claim 16, wherein the support has a calcination history of being exposed to a temperature of 1460 to 1700° C.

18. The shaped catalyst body according to any claim 16, wherein the shaped catalyst body comprises silver in an amount of 8 to 40 wt.-%, relative to the total weight of the shaped catalyst body.

19. The shaped catalyst body according to claim 16, wherein the shaped catalyst body comprises rhenium in an amount of 200 to 2000 ppm, relative to the total weight of the shaped catalyst body.

20. The shaped catalyst body according to claim 16, wherein the shaped catalyst body comprises a further promoter selected from the group consisting of cesium, potassium, lithium, tungsten, sulfur, and combinations thereof.

21. The shaped catalyst body according to claim 16, obtained by depositing silver and rhenium on a porous alpha-alumina support having a BET surface area of 1.7 to 10 m²/g and a calcination history of at least 1460° C.

22. The shaped catalyst body according to claim 16, the porous alpha-alumina support being obtained by
i) preparing a precursor material comprising a transition alumina and/or an alumina hydrate;
ii) forming the precursor material into shaped bodies; and
iii) calcining the shaped bodies at a temperature of at least 1460° C. to obtain the porous alpha-alumina support.

23. The shaped catalyst body according to claim 22, wherein the precursor material comprises at least 50 wt.-% of transition alumina.

24. The shaped catalyst body according to claim 22, wherein the precursor material comprises at most 30 wt.-% of alumina hydrate.

25. The shaped catalyst body according to claim 22, wherein the transition alumina has a loose bulk density of at most 600 g/L, a pore volume of at least 0.7 mL/g, as determined by nitrogen sorption, and a median pore diameter of at least 15 nm, as determined by nitrogen sorption.

26. The shaped catalyst body according to claim 22, wherein the transition alumina comprises a phase selected from gamma-alumina, delta-alumina and theta-alumina.

27. The shaped catalyst body according to claim 22, wherein the transition alumina comprises at least 50 wt.-% of a transition alumina having an average particle size of 10 to 100 μm based on the total weight of transition alumina.

28. The shaped catalyst body according to claim 22, wherein the alumina hydrate comprises boehmite and/or pseudoboehmite.

29. A porous alpha-alumina catalyst support having a BET surface area of 1.7 to 10 m²/g, the porous alpha-alumina catalyst support being obtained by
a) preparing a precursor material comprising a transition alumina and/or an alumina hydrate;
b) forming the precursor material into shaped bodies; and
c) calcining the shaped bodies at a temperature of 1460° C. to 1700° C. to obtain the porous alpha-alumina support.

30. A process for producing ethylene oxide by gas-phase oxidation of ethylene, comprising reacting ethylene and oxygen in the presence of a shaped catalyst body according to claim 16.
